(19) United States
(12) Patent Application Publication
(10) Pub. No.: US 2008/0234534 A1
(43) Pub. Date: Sep. 25, 2008

(54) Magnetic Therapeutic Appliance and Method for Operating Same

(75) Inventors: Philip Mikas, Brunnthal (DE); Georgios Alexandros Mikas, Gottingen (DE)

Correspondence Address: BROOKS KUSHMAN P.C., 1000 TOWN CENTER, TWENTY-SECOND FLOOR, SOUTHFIELD, MI, 48075, US

(73) Assignee: Philip Mikas, Brunnthal (DE)

(21) Appl. No.: 12/092,479

(22) PCT Filed: Oct. 31, 2006
(86) PCT No.: PCT/EP06/10484

§ 371 Date: May 2, 2008

(30) Foreign Application Priority Data
Nov. 2, 2005 (DE) 10 2005 052 152.5

(51) Int. Cl. A61N 2/04

(52) U.S. Cl. 600/14

(57) ABSTRACT

The invention relates to a therapeutic appliance for treating a patient with an electric field or a magnetic field. In known therapeutic appliances, resistance losses in the coils needed for the magnetic field lead to undesired warming of the therapeutic appliance. According to the invention, the energy of the coil (17) is carried off via a load resistor that can be arranged on the side away from an active area of the therapeutic appliance. Alternatively, or in addition to this, the energy of the coil (59) can be carried off via a return to the mains. Moreover, the invention proposes generating a magnetic field of variable magnitude with an oscillating circuit (59) which is cyclically, periodically or intermittently excited before oscillations can completely dissipate. Finally, the invention proposes the use of a magnetic core made of an iron powder.

The invention relates to a method for operation of a therapeutic appliance, in which a changing field is produced in a working area for therapeutic treatment of living tissue, in particular as claimed in the precharacterizing clause of claim 1 or the precharacterizing clause of claim 15. The invention also relates to a therapeutic appliance such as this, as claimed in the precharacterizing clause of claim 24 or of claim 36.

### PRIOR ART

For some time, living tissues have been treated with electrical or magnetic fields, for example for the treatment of nerve, bone or muscular illnesses in human beings. As is known from the dissertation “Grundlagen der Elektroklimatologie” [Principles of electro-climatology] by Dr. Ludwig, Freiburg, 1967, the healing processes which take place in living tissue are significantly based on changes in the fields. This results in the following requirements for therapeutic appliances:
The rate of change of the field should be as high as possible in order that the eddy currents which are induced in the body are high, with these in turn creating as much ion transport as possible in the tissue, by means of which the healing effects of the pulsed fields is intended to be justified.
A maximum flat density value that is as high as possible should be produced in order to ensure that the field penetrates as deeply as possible into the body of the living tissue.

DE 26 32 501 A1 discloses a therapeutic appliance in which a resonant circuit which is formed by a coil and a capacitor and can be interrupted by a make contact, a vacuum contact or a semiconductor port, is connected to a DC voltage source via a resistor and a diode. In order to operate the therapeutic appliance, the capacitor in the resonant circuit is first of all charged with the make contact open. The magnetic field in the area of the coil is used to treat the patient for a first charge-reversal pulse with the make contact being closed. The first charge-reversal pulse is followed by a number of ringing oscillations. The make contact is then opened again, in order to recharge the resonant circuit. One to ten individual pulses at intervals of one to ten seconds can be used for therapeutic purposes. In order to increase the depth effect of the magnetic lines of force, an essentially U-shaped ferromagnetic iron core is arranged in the coil, and its pole shoes are brought into contact with the body of the living tissue to be treated.

DE 39 25 878 A1 discloses a therapeutic appliance in which magnetic fields are used on which, in addition to an excitation frequency, one or more harmonics are also superimposed, with the aim of achieving an improvement in the effect of the magnetic field therapy. Such superimposition is achieved by the magnetic coil being part of a damped resonant circuit into which energy is introduced cyclically and in which, after the energy has been introduced, transient oscillations decay completely before the start of a subsequent cycle. The therapeutic appliance is intended to be powered by a small battery or rechargeable battery with a voltage, for example, of 6.9 or 12 V. Switching elements in the form of current transistors are operated such that the resonant circuit
is blocked for one millisecond, during which the components in the resonant circuit are connected to the electrical power supply, and
is used as separate resonant circuit for 999 milliseconds, in which oscillations which are produced as a consequence of the energy introduced into the resonant circuit can decay completely.

The coil has an inductance of 5 mH, while the bipolar capacitor is composed of two electrolytic capacitors, each of 4.5 mF. An NPN-6-75 transistor and a PNP-6-76 transistor are used as the transistors.

DE 699 10 590 T2 discloses a control device which uses a measured impedance value of the living tissue as the basis to apply a function generator or waveform generator to the therapeutic appliance that is suitable to bring about a desired treatment result.

DE 101 48 988 A1 discloses the principle of using switching transistors with a high-impedance input, so-called MOSFETs, for therapeutic appliances.

DE 100 54 477 A1 relates to the simultaneous application of a magnetic field and of an electric field to living tissue, with possible signal forms, changes in the fields and operating conditions for the fields matched to the respective constitution of the living tissue being disclosed.

DE 41 32 428 A1 discloses the simultaneous use of a plurality of coils in order to produce a magnetic field.

Further prior art is known, for example, from WO 2004/067090 A1, DE 196 33 323 A1, DE 197 08 542 A1 and DE 203 06 648 U1.

### OBJECT OF THE INVENTION

The present invention is based on the object of proposing a method for operation of a therapeutic appliance, as well as a therapeutic appliance, which is improved in terms of
the flux density change,
the maximum flux density values,
the heating of the therapeutic appliance,
the maximum possible operating duration with the maximum permissible heating, and/or
the energy consumption for operation of the therapeutic appliance.

### Solution

According to the invention, the object of the invention is achieved by the method according to the features of independent claim 1. A further solution to the problem on which the invention is based is provided by a method corresponding to the features of claim 15. A therapeutic appliance to achieve the object of the invention results corresponding to the features of claim 24. A further therapeutic appliance to achieve the object of the invention results corresponding to the features of claim 36. Further refinements of the invention follow from the dependent claims 2 to 14, 16 to 23, 25 to 35 and 37 to 39.

### DESCRIPTION OF THE INVENTION

The present invention is based on the discovery that, in known therapeutic appliances, the energy introduced into the coils is at least partially converted into heat in the resistance of the coil. This leads to the coils being heated severely even after a relatively small number of pulses. In consequence, in the worst case, temperatures above 130° C. can be produced in the therapeutic appliance and can lead to destruction of varnish insulation and/or of soldered joints. However, even temperatures below a limit temperature such as this may be undesirable. Experiments with known therapeutic appliances have shown that the temperature of a coil and of adjacent components can increase to more than 41° C. even after a small number of pulses, for example after approximately 100 to 500 pulses, depending on the pulse energy and the coil mass. For a therapeutic product licensed in Germany, the surface temperature of the therapeutic appliance must not exceed 41° C. in a working area that is operatively connected to the patient, since otherwise there would be a risk of skin burning. One consequence of this requirement is that the known therapeutic appliances must be switched off for a cooling-down phase on reaching the temperature of 41° C. In order to delay or to avoid such switching off, known therapeutic appliances reduce the frequency of the pulses to values of about 0.2 Hz, so that a pulse is produced only approximately every 5 seconds. In practice, this means that long application times, in particular of more than 2.5 hours, result for an entire body treatment, including the cooling-down times, for a predetermined total number of pulses.

As a further remedial measure, it is known for a large copper mass, in particular about 1 kg of copper wire, to be used to increase the time period for the coil to be heated as a result of the resistance, thus making it possible to increase the pulse repetition frequency for a certain time period to more than 10 Hz. As a further remedial option, it is known for a high surface temperature in the working area to be reduced by suitable insulation. However, insulation such as this increases the distance between the component producing the treatment field and the surface of the skin, resulting in a reduction in the penetration depth of the magnetic field into the body to be treated, and/or increased power requirements.

Furthermore, the invention has identified the fact that the rates of change of the fields that are produced in the known therapeutic appliances are limited, so that the eddy currents which are induced in the body and are responsible for the treatment result are also in some circumstances restricted:
The time of rise in known therapeutic appliances depends mainly on the magnitude of the voltage which is present when the pulse is applied to the coil. Because of the high peak currents of up to 150 A which have to flow through the coils in order to produce a desired strong magnetic field, capacitors are used as a voltage source for this purpose, and must be charged to 450 V before each pulse.
A freewheeling diode is often connected in parallel with the coil and protects a (semiconductor) switching element against the high induction voltage which would occur without the diode, as soon as the coil current decays. This freewheeling diode results in the decay time of the current pulses being relatively long, with the current decreasing in accordance with an exponential function whose time constant T is calculated solely from the ratio of the inductance L to its relatively low loss resistance R (τ=L/R).

On the basis of these considerations, the invention proposes the use of an electrical resonant circuit with (at least) one coil and (at least) one capacitor. The resonant circuit is supplied with energy from a power supply. An electrical or magnetic field which changes in an oscillatory form is produced in the coil and/or the capacitor. This field passes through a working area of the therapeutic appliance, in the area of the which the field is applied to the body area of the living tissue to be treated. By way of example, the working area is a fixed contact surface or a separate deformable contact body such as a therapy mat, in which case one or more working areas can be used, with one or more fields.

In the method according to the invention, an energy supply is activated by supplying energy to a component in the resonant circuit, such as the coil or the capacitor, through the power supply.

In a next method step, the energy supply from the power supply to the resonant circuit is deactivated, in particular by decoupling the power supply from the resonant circuit by means of a suitable switching element.

While the resonant circuit was preferably interrupted during the abovementioned method steps by means of a suitable switching element, transient oscillations of the resonant circuit are allowed in a subsequent method step. This allows the advantageous characteristics of a resonant circuit to be made use of according to the invention:
even without any external action, the profiles of the electrical variables in the resonant circuit are predetermined by the dynamic characteristics of the resonant circuit, in particular by the resistance in the resonant circuit, the capacitance and the inductance.
The frequency of the oscillation of the magnetic field can be predetermined in the design process by the choice of the inductance and the capacitance, with the frequency being correlated with the rate of change of the field, and therefore with the therapeutic effect produced in the living tissue. During the phase of the method with transient oscillations, there is no need for any complex external control to preset the desired electrical signals in the coil, in some circumstances. On the other hand, the damping of the transient oscillations can be predetermined by presetting the resistance R in the resonant circuit.

While, according to DE 39 25 878 A1, the transient oscillations decay completely, the invention has identified the fact that, as the transient oscillations decay to an increasing extent, the maximum value of the flux density falls, thus resulting in an increasing reduction in the effect of the therapeutic appliance in the body of the living tissue. In some circumstances, this results in particular risks, since the body is subjected during a treatment to different flux densities, flux-density changes and treatment durations, at different depths. Furthermore, despite the reduced effect as the transient oscillations decay, ever more power is lost in the coil, leading to heating of the therapeutic appliance. In summary, this means that, for such a complete decay of the transient oscillations, the ratio of the effect achieved in the body to the power loss in the form of heat developed in the coil is relatively poor.

According to the invention, the transient oscillations of the resonant circuit are therefore ended deliberately (before they have decayed completely) by interrupting the resonant circuit. Instead of having to wait until the energy in the resonant circuit has decayed completely, which would mean that it would be necessary to accept the heating of the coil and possibly of a damping resistor associated with this, the energy is dissipated from the resonant circuit at a time close to the end of the transient oscillations, via a component which is arranged separately from the resonant circuit. In consequence, the components which are used in the resonant circuit are used primarily to produce the therapeutic effect, while a different component can be used to dissipate the energy. This results in functional separation of the abovementioned components, thus allowing the components to be designed specifically for the respectively desired function, and avoiding aim conflicts. For example, this means that it is possible to arrange the component which is responsible for dissipation of the energy physically separately from the resonant circuit and thus at a distance from the working area where, for example, greater heating can be accepted or specific cooling measures can be adopted without adversely affecting the physical configuration of the working area.

The energy is preferably dissipated from the resonant circuit when the current through the coil and the flux density are in the region of a maximum. In the situation in which the remaining energy contained in the magnetic field of the coil is consumed both in the component which is responsible for the dissipation of the energy, in particular a load resistor, and in the resistance of the coil, the heat losses are distributed to a greater extent in the load resistor, the higher its resistance is in comparison to the resistance of the coil.

According to one development of the invention, the energy is dissipated from the resonant circuit (at least not exclusively) by conversion of heat in the area of a load resistor and the components of the resonant circuit, but at least partially by energy being dissipated from the resonant circuit by being fed back into the mains power supply. For this purpose, by way of example, the coil is connected by means of (semiconductor) switching elements to the mains power supply voltage such that this opposes the induced voltage in the coil. In consequence, the energy in the coil is not converted to heat in the coil or in an external load resistor. Once the feedback has resulted in the current in the coil decaying to zero, the coil can, for example, be disconnected from the mains power supply voltage again, and/or energy can once again be supplied from the mains power supply to the resonant circuit.

On the basis of a further method according to the invention, the method step of ending the transient oscillations is carried out before the energy in the transient oscillation has decayed to less than 50%, for example 75% and in particular 90%. This criterion can be checked, for example by:
detection of the actual energy in the resonant circuit, which can be done by monitoring the amplitude of the oscillation of the current and/or voltage, or
ending the transient oscillations after a predetermined number of cycles of the oscillations or a predefined time period.

This refinement of the invention makes it possible to deliberately make exclusive use of the area of the oscillation whose amplitude is adequate.

The transient oscillations are preferably ended after one cycle period of the resonant circuit, such that the state at the start of the transient oscillations, possibly with minor losses resulting from damping, is approximately recreated. According to one alternative refinement, the transient oscillations are ended approximately after half the cycle period of the resonant circuit, within which the magnetic field has been built up on the one hand and has decayed again.

The process of carrying out individual method steps that have been mentioned can be “triggered” by detection of an electrical signal whose evaluation, for example with regard to a threshold value being overshot or undershot, indicates the need to carry out the method step. For example, the current in the coil is detected in order to determine the time to end the transient oscillations, and is compared with a threshold value which is correlated with a desired maximum value. Alternatively or additionally, an electrical signal relating to the energy supply from the power supply to the component in the resonant circuit can be detected in order to determine the time for deactivation of the energy supply and/or to allow transient oscillations of the resonant circuit.

An alternative or additional option for determination of the times to carry out at least one method step, in particular the method step of ending the transient oscillations, is for transient oscillations to be allowed for a defined time period, which is preferably correlated with the cycle period of the oscillations of the resonant circuit.

When the aim is to repeatedly apply pulses in order to reinforce the effect of the therapeutic appliance, the method steps of the method according to the invention can be carried out cyclically. Since the heat produced in the therapeutic appliance is less than that in known appliances, the method steps can also be carried out cyclically at a frequency which, in particular, is higher than 5 Hz or even 10 Hz, thus making it possible to reduce the treatment time, in some circumstances, without changing the treatment result.

An AC voltage mains power supply is advantageously used to supply energy from the power supply to the components in the resonant circuit, so that there is no need for a low-voltage DC voltage source. Phase gating control can be connected between the components in the resonant circuit and the AC voltage mains power supply, deliberately making use of subareas of the AC voltage for application to the components in the resonant circuit, in particular those subareas in the area around the maximum of the mains power supply voltage, thus allowing short energy supply times.

The resonant circuit, in particular the capacitor in the resonant circuit or the load resistor, is preferably connected to the circuit GND potential. This has the advantage that a switching element which is responsible for the energy supply between the power supply and the resonant circuit has only the induced voltage in the coil applied to it and not also the supply voltage, thus resulting in a reduced voltage load on this switching element. This makes it possible to use less costly switching elements. Furthermore, when the public 230 V mains power supply is used as the voltage source, high interference voltage pulses can be expected which, by virtue of the refinement according to the invention, cannot act on the abovementioned switching element and destroy it, thus considerably improving the circuit reliability.

The approach described above is based on the assumption that a subsequent cycle is started after the energy in the resonant circuit has being dissipated, with the consequence that remaining residual energy in the resonant circuit is dissipated in a suitable manner since this can no longer be used for any worthwhile therapeutic process, taking into account the thermal budget. In the case of a further solution to the problem on which the invention is based, a “forced” oscillation is maintained in the resonant circuit by supplying energy from the power supply to the resonant circuit cyclically, periodically or intermittently.

Depending on the circumstances, this leads to the following advantages:
The resultant oscillation can be predetermined by the choice of the resonant circuit excitation. For example, the resultant oscillations may be composed of transient oscillations at the natural frequency of the resonant circuit and forced oscillations at an excitation frequency, thus making it possible to achieve the therapeutic effect with a plurality of frequencies. On the other hand, the excitation frequency may be deliberately chosen, and in some circumstances may be varied depending on the patient or the state of the patient, or else may be varied over a treatment of the patient, thus making it possible to achieve finer tuning of the treatment of the patient.
While, according to other solutions, energy must be deliberately dissipated or destroyed, the energy can be supplied to the resonant circuit in such a way that only the energy which is dissipated over one cycle of the oscillation of the resonant circuit need be supplied again by the power supply in order to produce a stable oscillation state in the resonant circuit. This makes it possible to reduce the power required from the voltage supply for the therapeutic appliance.
When using a forced oscillation, variable or decaying amplitudes can in some cases be avoided. Instead of this, a more or less constant amplitude and/or at least a frequency can be produced deliberately in the resonant circuit.

Any desired signals may be used to excite the resonant circuit via the power supply, for example
stochastic
non-cyclic,
cyclic signals
   at one or more frequencies, for example harmonics or sub-harmonics of a fundamental frequency, in which case the oscillation that is maintained may be cyclic or non-cyclic, provided that the electrical signals of the oscillation, for example the current in the coil, reach a magnitude that is required for the therapeutic purpose, at least at times.

The use of a harmonic signal from the energy supply for the resonant circuit is particularly advantageous for production of a regular signal in the resonant circuit.

The energy to be introduced into the resonant circuit can be minimized by the frequency of the excitation signal corresponding approximately to the resonant frequency of the resonant circuit since the production of large amplitudes of the electrical signals in the resonant circuit allows resonant operation for small excitation amplitudes.

In a further refinement of the method according to the invention, this is cyclic, with a constant duration for cyclic processes or a variable duration. A switching element can be operated at an operating time within one cycle, resulting in the resonant circuit being interrupted. In a first phase within a cycle before the time at which the switching element is operated, a transient oscillation of the resonant circuit is permitted, with the advantages mentioned above. The time duration of the first phase is, for example, one quarter, one half, three quarters of one cycle period of the free oscillation of the resonant circuit, or 1.5 times, twice, 2.5 times, three times, etc the cycle period of the free resonant circuit, so that the electrical states in the resonant circuit at the start of the first phase can correspond approximately to the state at the end of the first phase, for example with it being possible for the start and/or the end of the first phase to occur in the region of an extreme of the energy in the coil or in the capacitor, or at a zero crossing thereof.

Furthermore, for one preferred refinement, energy can be supplied to the components in the resonant circuit within one cycle in a second phase after the time at which the switching element can be operated, with the resonant circuit interrupted. By way of example, the time duration of the second phase may be predetermined a priori or from a family of characteristics, which can be designed on the basis of how much energy must be supplied to the resonant circuit, which currents are permissible to produce the energy, what energy supply source is available, etc. In an alternative or additional refinement, an electrical variable of a component in the resonant circuit may be detected, for example the current in a coil and/or the voltage across the capacitor, in which case the end of the second phase may be indicated by a threshold value of the monitored variable being exceeded.

According to a supplementary proposal of the invention, the energy state of the components in the resonant circuit is left essentially constant within one cycle in a third phase after the time at which the switching element is operated, with the resonant circuit interrupted. In this case, essentially constant means a switching state in which the electrical connections of the components are very largely interrupted and the energy levels in them change only insignificantly. In the third phase, for example, not only can the resonant circuit be interrupted but the components in the resonant circuit can also be disconnected from the power supply. The phases (first phase, second phase, third phase) that have been mentioned may follow one another in any desired sequence.

In a further solution to the problem on which the invention is based, a therapeutic appliance which is used in particular to carry out one of the abovementioned methods is equipped with a ferromagnetic core passing through the coil, or a magnet core composed of magnetic powder, or iron powder. In principle, a magnet core leads to reinforcement of the magnetic field. This means that the current level required to produce a predetermined magnetic field that is required to produce the therapeutic effect can be reduced, thus leading to a reduction in the resistive losses, which are proportional to the square of the current, and thus to a reduction in the heat that is developed. An iron core composed of ferromagnetic powder allows the magnetic field to be changed at fast rates without this resulting in eddy currents, and the eddy-current losses associated with them, in the iron core. Furthermore, magnet cores composed of a ferromagnetic powder can also be produced in a simple manner at low cost, in some circumstances with any desired external geometry. This offers particular configuration options for example in the contact area of a magnet core with the patient in the working area since any desired magnet cores and pole shoes can be manufactured here.

While the strength of the magnetic field for a coil without an iron core decreases continuously radially outwards, the flux density in an iron core can influenced and deliberately predetermined by predefining the geometry of the iron core and the contact surface area with the working area such that, for example, the flux density extends in a more or less constant form over a larger area.

The saturation flux density of a magnet core composed of a ferromagnetic powder of >0.5 Tesla (in particular >1.0 Tesla) is preferably used, so that the therapeutic appliance is designed to be highly effective, with high flux densities.

In a further refinement of the invention, the therapeutic appliance has a control device, for example in the form of a microcontroller, which controls switching elements in order to allow different operating phases of the therapeutic appliance, preferably corresponding to the abovementioned method. In this, the following items can be provided in the control device:
time control,
closed-loop control with measurement variables being fed back, and/or
selection of suitable times for monitoring individual electrical signals.

The safety of the therapeutic appliance and compliance with the legally stipulated requirements can be improved by providing a temperature sensor, for example in the area of the coil or in the working area, in the therapeutic appliance. The measurement signal from the temperature sensor is passed to a monitoring unit which, for example, is formed integrally with a microcontroller. The monitoring unit monitors the measured value of the temperature sensor. If the temperature sensed by the temperature sensor exceeds a threshold value, the therapeutic appliance can initiate suitable measures, for example by producing a fault signal for the user of the therapeutic appliance, in particular in the form of a warning lamp or an audible signal, or can act on the electrical states in the coil, the resonant circuit and/or the power supply and its coupling to the coil in order to cause the therapeutic appliance to cool down or to avoid further heating. In addition, an overtemperature switch can be fitted to the coil and ensures, if the temperature monitoring unit fails, that the electrical power supply to the coil is mechanically interrupted if the coil temperature becomes too high.

The capacitor in the resonant circuit is subject to withstand voltage and capacitance requirements which in some cases result in high component costs. If a component with a very high withstand voltage is used, a very large number of components, for example more than 70 components, must be used because of the relatively low capacitance of components such as these. In addition, certain components exist only in specific standard values, so that it will not always be possible to extract the maximum power from the therapeutic appliance. According to the invention, therefore, the capacitor in the resonant circuit is formed using a multiplicity of relatively low-cost, high-capacitance film capacitors, for example with a capacitance in the region of several microfarads, each with a relatively low withstand voltage, and which are connected to one another in parallel and/or in series so as to achieve a desired capacitance value with the required withstand voltage.

In a further solution to the problem on which the invention is based, a therapeutic appliance has a control device. The control device is connected to at least one switching element via signal connections. The resonant circuit is closed in a first phase for the operated position of this switching element.

The control device is also connected via the same or other signal connections to the same or to another switching element. When this switching element is operated by the signal connection, the resonant circuit is opened in a second phase, allowing energy to be supplied between the electrical power supply and the at least one component in the resonant circuit.

In order to ensure that the transient oscillations in the resonant circuit do not decay completely in the first phase, the control device has a means which is suitable for determining a time at which the first phase of the transient oscillations must be ended. This time is determined in the control device such that transient oscillations in the resonant circuit do not decay below a predetermined level, for example half the amplitude, 80% of the amplitude or 90% of the amplitude.

In the simplest case, said means is a time control, which presets the end of the first phase to be fixed, or as a function of operating parameters or measured values, on the basis of a family of characteristics or a mathematical function. It is likewise possible to detect the transient oscillations directly or indirectly and to compare the transient oscillations with a predetermined level or threshold value by means of a suitable algorithm in the control device.

Furthermore, the same or another switching element can operated via the same or other signal connections, with the resonant circuit being interrupted with such operation in the third phase, and the components in the resonant circuit being decoupled from the voltage supply. A third phase such as this is used in particular to prevent further heating of the therapeutic appliance, and if necessary to cool it down by convection.

For one particular refinement of the therapeutic appliance, the resonant circuit has different paths for different current directions, with a component which blocks in one current direction being arranged at least in one path such that the other path is used for current running in this direction. A switching element is arranged in the other path by means of which, for example, it is possible to switch from one phase to another phase (first phase, second phase, third phase). The switching element in the second path is operated by means of the control device at a time at which the electrical signals of the transient oscillation also at least run via the first path. This refinement is based on the discovery that operation of the switch for a switching process in a very heavily electrically loaded path could cause a high induced voltage, which in some circumstances destroy a semiconductor switch, in the coil of the resonant circuit. This is prevented by the parallel path. Furthermore, in combination with further features according to the invention, a first phase can be ended in a simple manner by opening the abovementioned switching element, when the second path is blocked because the switching element is open and the electrical variables in the resonant circuit have changed such that the first path is also blocked because of blocking by the component in one direction.

In a further refinement of the invention, the power supply or voltage supply is a high-voltage supply. A high-voltage supply such as this may be designed for a wide input voltage range so that the circuit can be operated from any desired mains power supply voltage and at any desired mains power supply frequency, in some circumstances with a downstream rectifier and filter electrolytic capacitor. The high-voltage supply or high-voltage transmission can also be designed for low voltage, therefore also allowing the therapeutic appliance to be operated using a 12 V power supply unit or a rechargeable battery. The use of a rechargeable battery is also made possible by the high efficiency made possible by the invention and the small amount of energy required by the therapeutic appliance.

Advantageous developments of the invention will become evident from the patent claims, the description and the drawings. The advantages, as mentioned in the introductory part of the description, of features and of combinations of a plurality of features are only by way of example and may be used alternatively or cumulatively without the advantages necessarily having to be achieved by embodiments according to the invention. Further features can be found in the drawings—in particular the illustrated geometries and the relative dimensions of a plurality of components with respect to one another and their relative arrangement and operative connection. The combination of features of different embodiments of the invention or of features of different patent claims is likewise possible in a different manner to the selected back-references of the patent claims, and is hereby proposed. This also relates to those features which are illustrated in separate drawings or are mentioned in their description. These features can also be combined with features from different patent claims. Features stated in the patent claims can likewise be omitted from further embodiments of the invention.

### BRIEF DESCRIPTION OF THE FIGURES

The invention will be explained and described in more detail in the following text with reference to preferred exemplary embodiments which are illustrated in the figures, in which:

FIG. 1 shows a therapeutic appliance according to the invention with a handheld part and a module housing.

FIG. 2 shows a schematic block diagram of an electrical circuit of a therapeutic appliance according to the invention.

FIG. 3 shows a circuit as part of a therapeutic appliance according to the invention for dissipation of energy from a coil via a load resistor located in the freewheeling branch.

FIG. 4 shows a circuit as part of a therapeutic appliance according to the invention, in which energy is dissipated from a coil via a load resistor which is connected to GND.

FIG. 5 shows the waveforms of a mains power supply voltage, of a current in a coil and of a voltage in a capacitor in a therapeutic appliance according to the invention, as shown in FIGS. 6 to 10.

FIG. 6 shows a further refinement of a circuit as part of a therapeutic appliance with a resonant circuit in a switching state in which an energy supply from the power supply to the coil in the resonant circuit is activated.

FIG. 7 shows the circuit shown in FIG. 6, in a switching state in which transient oscillations are allowed in the resonant circuit.

FIG. 8 shows the circuit as shown in FIG. 6, in a switching state correlated with that shown in FIG. 7, with the transient oscillations being illustrated with opposite current profiles to those shown in FIG. 7.

FIG. 9 shows the circuit as shown in FIG. 6, illustrating one phase of the transient oscillations after returning to a current profile as shown in FIG. 7.

FIG. 10 shows the circuit as shown in FIG. 6, in a switching state in which the energy in the resonant circuit is dissipated by being fed back into the mains power supply.

FIG. 11 shows a schematic block diagram with two alternatives for a method according to the invention.

FIG. 12 shows a further refinement of a circuit as part of a therapeutic appliance with a resonant circuit and an energy supply via a high-voltage transformer.

FIG. 13 shows the waveforms of a capacitor voltage and of a coil current for a circuit as shown in FIG. 12.

FIG. 14 shows a further circuit as part of a therapeutic appliance with two different paths for free transit oscillations in the resonant circuit, and with a switching element with three switching states.

FIG. 15 shows the waveforms of a voltage in a capacitor and a current in a coil for a circuit as shown in FIG. 14.

FIG. 16 shows one embodiment of the circuit as shown in FIG. 14, with MOSFET transistors.

FIG. 17 shows a schematic block diagram of an electrical circuit for a therapeutic appliance according to the invention.

### FIGURE DESCRIPTION

In some cases, components whose function and arrangement in a circuit correspond are provided with the same reference symbols in the following figures, with the use of a component in different exemplary embodiments being identified by different letters added to the reference symbol.

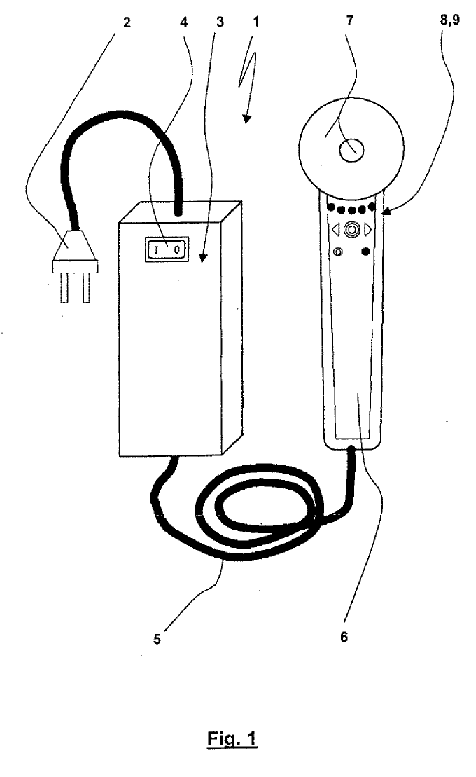

FIG. 1 shows a therapeutic appliance 1 with a plug 2 for connection to a public electrical power supply system, and a module housing 3 with a mains power supply switch 4, which is connected to a handheld part 6 via a connecting cable 5. The handheld part 6 has a working area 7 in the area of which the handheld part 6 is operatively connected to a patient to be treated. For this purpose, the working area 7 may be placed directly on the skin of the patient. The handheld part 6 has control elements 8, for example in the form of knobs, switches or slides, as well as indicators 9, for example lamps, LEDs or the like. Contrary to the embodiment illustrated in FIG. 1, control elements 8 and indicators 9 may alternatively or additionally be provided in the area of the module housing 3. The electrical circuit and the power supply can be influenced by the control elements 8 for matching to the respective requirements, while the indication 9 provides the user of the therapeutic appliance 1 with feedback about the operating mode and any fault messages.

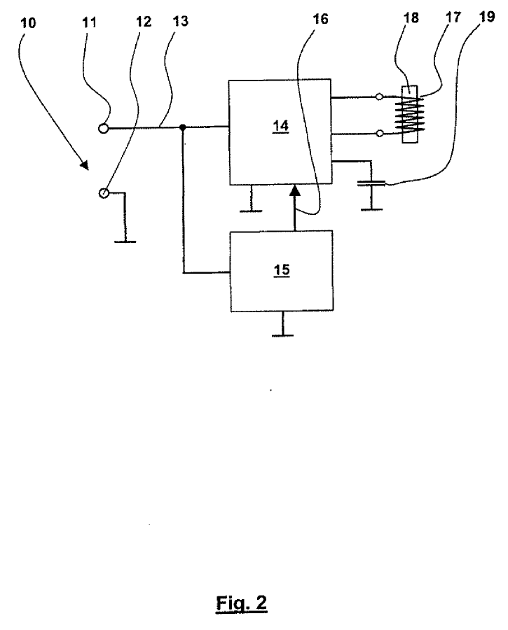

FIG. 2 shows a schematic block diagram of the therapeutic appliance with a voltage supply 10 which, for example, is a 230 V AC voltage source at a frequency of 50 Hz. The voltage supply 10 has a pole 11 (L1) and a pole 12 (N). The voltage supply 10 is connected via electrical connections 13 both to a power output stage 14 and to control electronics 15. The control electronics 15 act on the power output stage 14 via a connection 16. The power output stage 14 is electrically connected to a coil 17 with a core 18, and to a capacitor 19. FIG. 3 shows a circuit 20 in which one pole of the voltage supply 10a is connected to GND via the coil 17a and a switching element 21a. A branch with a load resistor 22a and a diode 23a is connected in parallel with the coil 17a. The diode 23a is in this case connected such that an induced current 25a flowing as a consequence of the induced voltage 24a can flow through the load resistor 22a. In the situation in which the supply voltage 26a of the voltage supply 10a is 200 V and an induced voltage of 24a of 800 V acts in the coil 17a, a voltage 30a acting on the switching element 21a is added to the sum of the induced voltage 24a and the supply voltage 26a that is to say 1000 V, for operation of the switching element 21a.

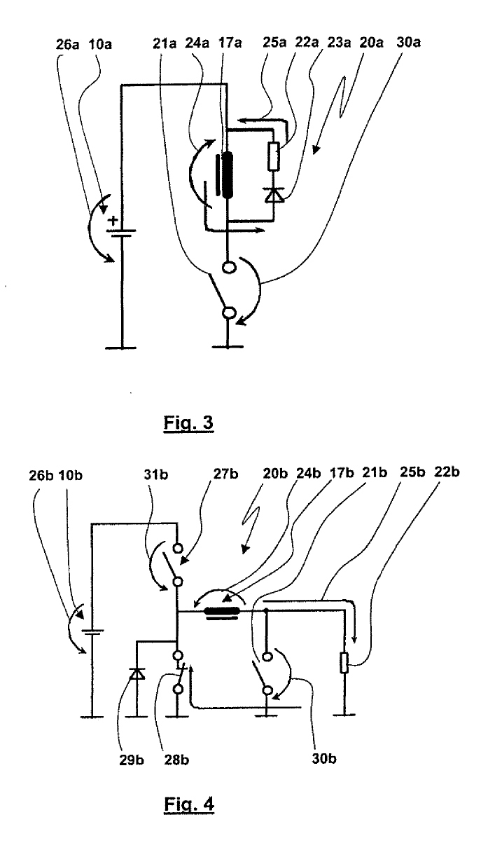

As shown in FIG. 3, the voltage supply 10b for the alternative circuit 20b illustrated in FIG. 4 is connected via a switching element 27b, a coil 17b and a switching element 21b to GND. The load resistor 22b is tapped off in a parallel circuit between the coil 17b and the switching element 21b, and is likewise connected to GND. A switching element 28b and a diode 29b are in each case connected in between in further parallel branches, which branch off between the switching element 27b and the coil 17b, with the anode of the diode 29b connected to ground. In an alternative refinement, contrary to FIG. 4, the branch with the switching element 28b is omitted.

The voltage 24b induced in the coil 17b is also dissipated via the induced current 25b in the load resistor 22b in the switching states as illustrated in FIG. 4 in which the switching elements 27b and 21b are open (and the switching element 28b may be closed). In this case, only a voltage 30b which corresponds to the induced voltage 24b acts on the switching element 21b. A voltage 31b which corresponds to the supply voltage 26b acts on the switching element 27b. Contrary to FIG. 3, the switching element 21b is therefore not subject to the supply voltage 26b and therefore to any interference voltage pulses, if the supply is provided via the mains power supply.

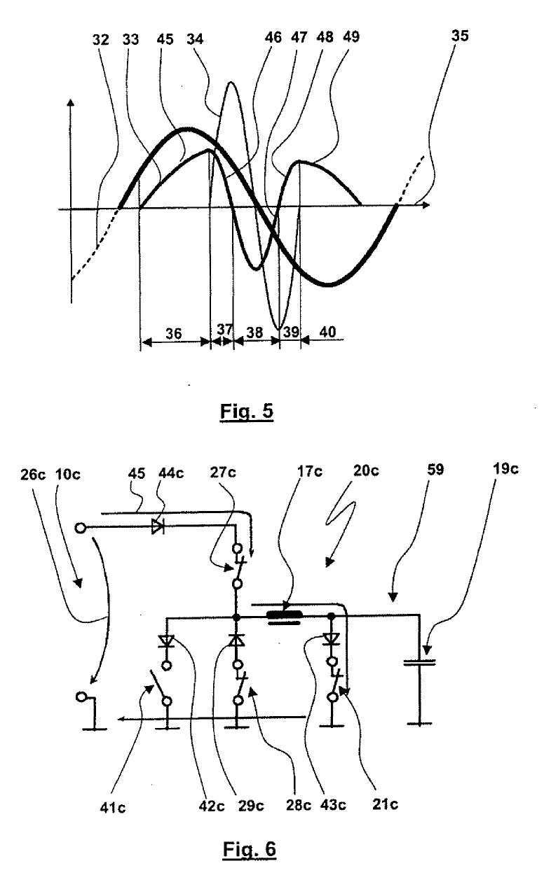

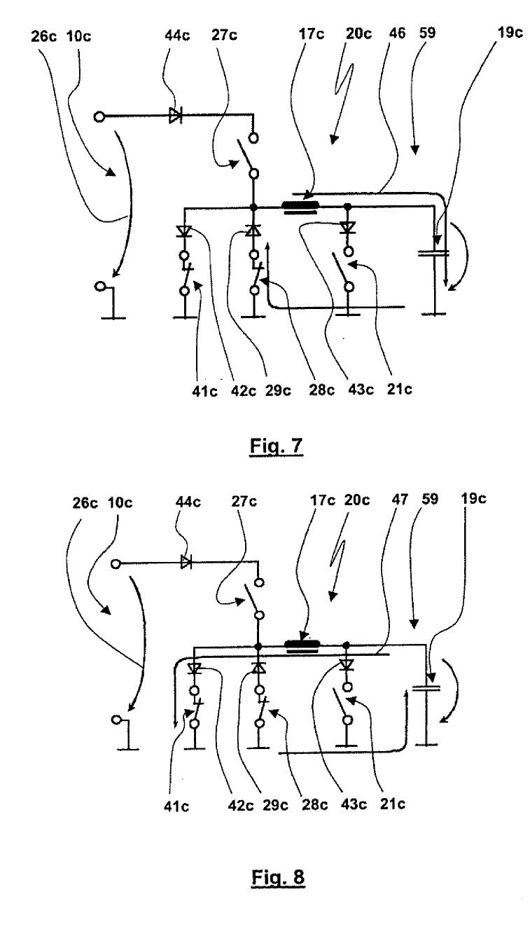

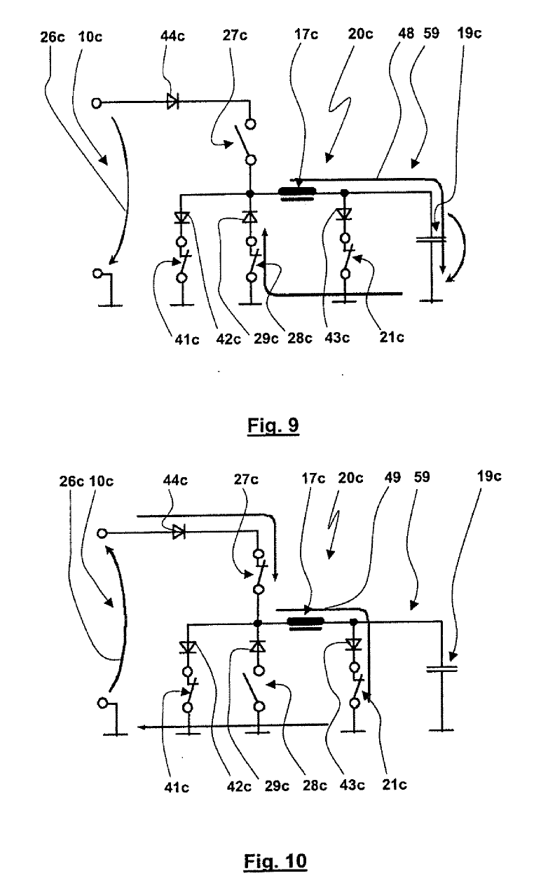

FIG. 5 shows the signals of a mains power supply voltage 32, of a current 33 in a coil 17 and the voltage 34 across a capacitor 19c plotted against the time 35 for the situation in which (contrary to FIGS. 3 and 4) the therapeutic appliance 1 has a resonant circuit 59. FIG. 5 does not show the magnetic field plotted against the time, since this exactly follows the waveform of the current 33 in the coil 17. In FIG. 5, a phase 36 is correlated with the switching state of switching elements of an alternative circuit 20c, as illustrated in FIG. 6. One phase 37 corresponds to the switching state illustrated in FIG. 7 with the illustrated orientation of the illustrated currents, while a phase 38 is correlated with the corresponding switching state, but with differently oriented currents as shown in FIG. 8. Phase 39 is correlated with the switching state illustrated in FIG. 9 and the illustrated flow directions of the currents, while the phase 40 shows energy being dissipated from the resonant circuit 59 into the mains power supply with the switching states and current profile directions illustrated in FIG. 10.

The circuit 20c illustrated in FIGS. 6 to 10 corresponds essentially to the circuit 20b shown in FIG. 4, but with the load resistor 22b replaced by the capacitor 19c, and with a switching element 28c being connected between the diode 29b and GND. In addition, a current path is provided for the opposite current direction in parallel with the series circuit formed by the diode 29c and the switching element 28c. This additional current path comprises a diode 42c and a switching element 41c. Furthermore, the switching elements 21c and 27c are preceded by a respective diode 43c, 44c.

In the phase 36 for the positive mains power supply voltage 32, the switching element 41c is opened, while the switching elements 27c, 28c and 21c are closed. This leads to a charging current 45, which passes through the diode 44c, the switching element 27c, the coil 17c, the diode 43c in its forward-bias direction and through the switching element 21c to GND. As the duration of the phase 36 progresses, the current rises as shown by the signal profile 33 and has reached its maximum at the end of the phase 36, thus predetermining the initial energy for the resonant circuit 59 formed in the phases 37, 38, 39.

In the transition area between the phases 36 and 37, switching takes place to the switch positions shown in FIG. 7, for which the switching elements 27c and 21c are open, while the switching elements 28c and 41c are closed. In order to ensure that the previous current is not interrupted on opening of the switching elements 21c and 27c, by which means a high induced voltage can be avoided, the switching element 28c must be closed before this opening process. The diode 29c in this case prevents the mains power supply voltage from being short-circuited via the switching elements 27c and 28c.

In the phase 37, the diode 42c is reverse-biased for the positive current 33, while the diode 29c is forward-biased, so that the switching element 28c, the diode 29c, the coil 17c and the capacitor 19c form a resonant circuit 59. An oscillating current 46 occurs in the phase 37.

In the transition area from the phase 37 to the phase 38 (see FIG. 8), the current 33 changes its direction, so that the diode 29c is reverse-biased in the phase 38, while the diode 42c is forward-biased. In this case, the resonant circuit 59 is formed by the switching element 41, the diode 42c, the coil 17c and the capacitor 19c, resulting in an oscillating current 47.

During the transition from the phase 38 to the phase 39, the current 33 once again changes its mathematical sign, so that the switching states, the resonant circuit 59 that is formed and the resultant electrical signals as shown in FIG. 9 essentially correspond to FIG. 7 and the associated description, with an oscillating current 48.

On the transition from the phase 39 to the phase 40, the resonant circuit 59 formed as shown in FIG. 9 is interrupted by opening the switching element 28c. This is preferably done at a time at which the capacitor voltage is approximately zero and the maximum current is flowing in the coil. Furthermore, the mains power supply voltage is in this case preferably negative. In this case, the voltage source 10c is connected to GND via the diode 44c, the switching element 27c, the coil 17c, the diode 43c and the switching element 21c. The voltage induced in the coil 17c is in the opposite sense to the voltage of the voltage supply 10c so that the resultant outflowing current 49 flows back into the voltage supply 10c, thus quickly dissipating the energy from the resonant circuit 59 and the coil 17c.

In order to carry out the method according to the invention, a check is first of all carried out in a method step 51 in a control device 50 to determine whether the supply voltage satisfies a predetermined criterion. In the situation in which it is found that the criterion is satisfied, this represents the initial point of the phase 36. The criterion is preferably chosen such that the mains power supply voltage during the phase 36 is as a high as possible and has no change in its mathematical sign. For example, a zero crossing of the mains power supply voltage may be chosen as the criterion and directly triggers the initiation of the phase 36, or triggers this with a time delay.

In a subsequent method step 52, energy is supplied for the phase 36 as shown in FIG. 6 to a component in the resonant circuit 59, to the coil 17c in the exemplary embodiment illustrated in FIGS. 6 to 10.

In a subsequent method step 53 a criterion is checked to determine whether sufficient energy has been built up in the components in the resonant circuit 59, in this case a sufficient current flow in the coil 17c. For example, a check is carried out to determine whether the current 45 has reached a predetermined threshold value. If the criterion is satisfied, the transition from the phase 36 to the phase 37 takes place. The time since the start of the phase 36 can be checked as an alternative or additional criterion, so that the phase 36 has a defined duration, irrespective of the electrical variables that occur.

In the transitional region to the phase 37, the resonant circuit 59 is closed in a method step 54, by allowing transient oscillations and maintaining these for the phases 37, 38, 39, with the states as shown in FIGS. 7, 8 and 9.

A check is carried out in method step 55 to determine whether the transient oscillations should be ended. A criterion to be checked in this case may, for example, be the decrease in the oscillations in the resonant circuit 59, in which case this decrease can be checked in an absolute form by a threshold value being undershot or, for example, in a relative form by comparison of an instantaneous amplitude with the initial amplitude. It is also possible to evaluate a time duration of the phases 37, 38, 39 as the criterion, such that these phases have a predetermined duration. Fractions of cycles, a multiplicity of oscillation cycles, half the oscillation period, one oscillation period, 1.5 or two oscillation periods may be used for the resultant oscillation of the resonant circuit. According to the illustrated embodiments, the criteria is also chosen such that the transient oscillations are ended at a time at which the voltage across the capacitor is approximately 0, and the current 48 is approximately a maximum, so that the energy in the resonant circuit is at least mainly stored in the coil 17c.

If the check in method step 55 shows that the free oscillations in the resonant circuit 59 should be ended, then the switching state shown in FIG. 10 is produced via the control device 50 in the method step 56, in which the energy stored in the coil 17c is fed back into the mains power supply.

After this, the method jumps back to the method step 51, and a further check can be carried out in a method step 57. For example, in the method step 57, the control device 50 can check whether the temperature conditions are satisfied or whether the method must be interrupted for a certain cooling-down time. Furthermore, a constant waiting time of several milliseconds can be provided in the method step 57. It is likewise possible to check further fault signals of the therapeutic appliance or any signals of the user of the therapeutic appliance.

For an alternative refinement of the method, instead of the method step 56, a switching state is brought about in a method step 58, in which the energy in the resonant circuit 59 is dissipated via an external load resistor.

The measures according to the invention allow the effectiveness of the therapeutic appliance to be increased by many times in comparison to conventional, known appliances. The application time for an entire body treatment may be reduced, for example from 2.5 hours for a known therapeutic appliance to 2 minutes now. In this context, effectiveness means the voltage/time integral induced per magnetic pulse in a coil, which is first of all rectified and is then electronically integrated over several 10 s of seconds.

The invention also proposes that a maximum flux density of 0.8 Tesla be achieved with less than 20 A rather than with 150 A as a in the case of the prior art. To do this, the number of turns is increased by a factor of approximately 2 or more in comparison to the number of turns on conventional coils. A coil with about 1700 windings (±200 windings) is used according to the invention.

Furthermore, an iron core, preferably composed of a ferromagnetic iron powder, can be used in the coil.

In the situation in which transient oscillations are terminated at the time of the coil current maximum, the switching elements involved can be protected.

Appropriate design of the resonant circuit 59 by choice of the inductance and of the capacitance makes it possible to produce steep pulse flanks, thus making it possible to increase the effectiveness per pulse. It is also possible for the inductance or the capacitance to be variable, in steps or continuously, thus allowing the frequency of the transient oscillations to be variable.

In order to introduce the initial energy into the resonant circuit 59, phase gating control can be connected between the coil and the supply voltage without having to previously charge a capacitor.

In addition, the embodiment with the resonant circuit 59 shown in FIGS. 6 to 10, the switching element 21c is subject only to the maximum capacitor voltage—and not additionally to the mains power supply voltage.

The switching elements 27, 41, 28, 21 are preferably semiconductor switches or MOSFET or IGBT transistors. In the situation in which IGBTs are used, the diodes 44, 29, 42, 43 may be omitted.

The resonant circuit 59 preferably has a resonant frequency at about 200 Hz±50 Hz, preferably 210 Hz+15 Hz.

FIGS. 12 to 17 show further exemplary embodiments of the invention, in which energy is supplied cyclically, the switching elements are operated cyclically, and there is a cyclic signal profile in a resonant circuit, in this case by way of example with a constant cycle period of one cycle and periodic equalization of the energy dissipated for transient oscillations, by means of intermittent coupling to the power supply.

In FIG. 12, a resonant circuit 60a is formed with a coil 61a and capacitor 62a which are connected to one another via a switching element 63a. Energy can be applied to the resonant circuit 60a via a high-voltage transformer 64, which is fed from a voltage source 65a. A further switching element 66a is connected between the resonant circuit 60a and the high-voltage transformer 64a.

FIG. 13 shows the operation of the circuit as shown in FIG. 12; first of all, the capacitor 62a is charged in an initial phase 67a, in which the switching element 63a is open and the switching element 66a is closed, via the voltage source 65a and the high-voltage transformer 64a. The voltage 68a across the capacitor 62a rises approximately continuously in the initial 67a. The end of the initial phase 67a is reached after a predefined time interval, or is reached when the voltage 68a has reached a predefined threshold value. At the time 69a for ending the initial phase 67a, the voltage source 65a and the high-voltage transformer 64a are deactivated, which can be done by opening the switch 66a. Approximately at the same time, the switch 63a is closed at the time 69a, so that the resonant circuit 60a is closed.

In the first phase 70a, which follows after the time 69a, transient, exponentially falling harmonic signals result for the voltage 68a across the capacitor and for the current 71a in the coil. After one cycle of the oscillation of the voltage 68a and of the current 71a, after which the current 71a in the coil is approximately zero again and the voltage 68a across the capacitor is a maximum again, possibly subject to the electrical losses, the switch 63a is opened at a time 72a, so that the energy exchange between the coil 61a and the capacitor 62a is interrupted.

For a possible method whose electrical signals are illustrated in FIG. 13, the switch 66a is closed at a time in the vicinity of the time 72a, in particular at the same time that the switching element 63 is opened, so that the voltage source 65a is once again connected to the coil 62a, with the interposition of the high-voltage transformer 64a.

In a second phase 73a, which occurs after the time 72a, the capacitor 62a is charged again, for example with an approximately continuously rising voltage from the capacitor 62a. At a time 74a at the end of the second phase 73a, the switch 66a is opened again, and the switch 63a is closed again, so that a first phase 70a is repeated, with a second phase 73a following it. A cycle with a first phase 70a and a second phase 73a with a cycle period 75a is repeated continuously in accordance with the desired therapeutic success.

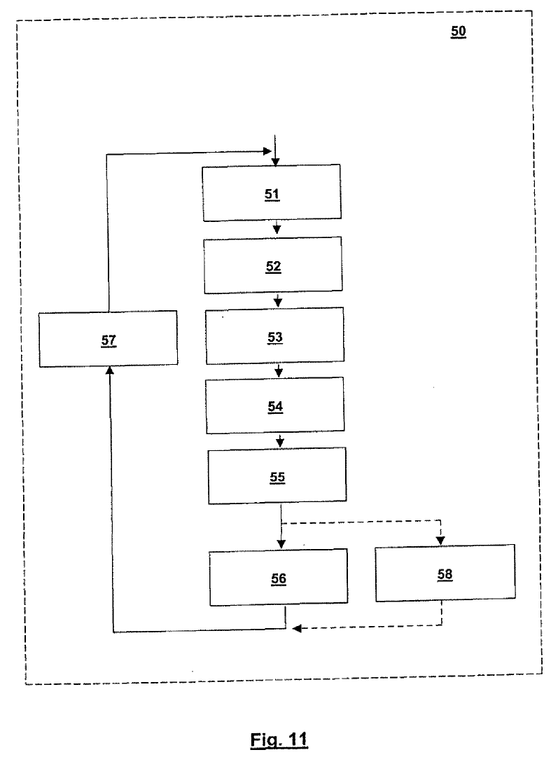

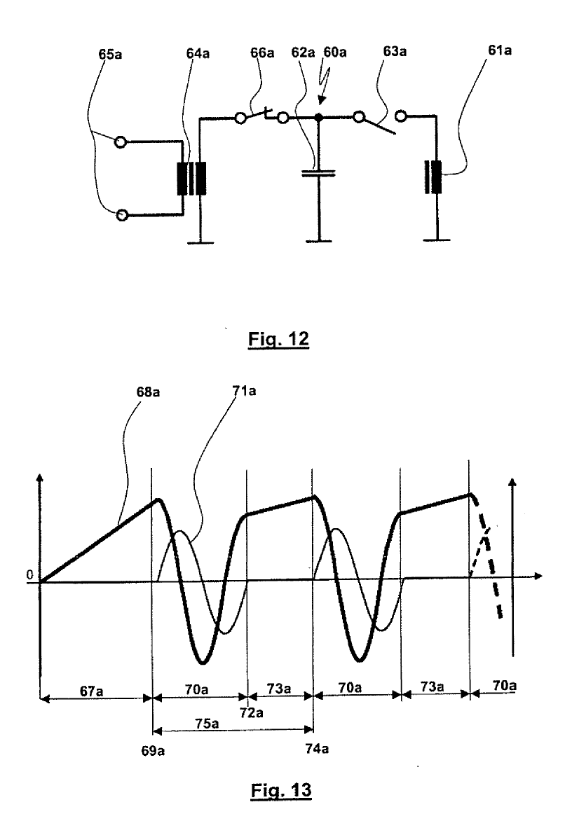

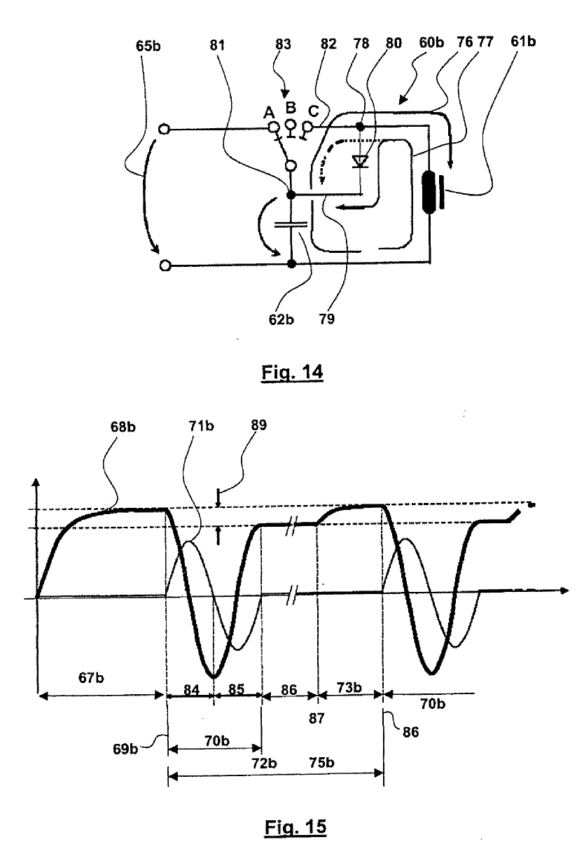

The current waveforms for different directions of the transient current are illustrated by the arrows 76 and 77 for the resonant circuit 60b, for the circuit according to the invention as illustrated in FIG. 14. For the current direction indicated by the arrow 77, the coil 61b is connected via the outgoer 78 and a path 79 to a diode 80, which is forward-biased in the direction of the arrow 77, and an outgoer 81 is connected to the capacitor 62b. When the current changes its direction as shown by the arrow 76 for transient oscillations of the resonant circuit 60b, then the diode 80 becomes reverse-biased. A parallel path 82 is connected between the outgoers 78, 81, with a switching element 83.

The switching element 83 has switch positions A, B, C, with the path 82 being closed in the switch position C in order to allow current to flow as shown by the arrow 76. In the switch position A, the switching element 83 interrupts the connection between the outgoers 78, 81 and at the same time makes a connection between the outgoer 81 and a voltage source 65b, possibly with the interposition of a high-voltage transformer 65b. In a middle switch position B, the outgoer 81 is connected neither to the outgoer 78 nor to the voltage source 65b.

For a method for operation of a therapeutic appliance having a circuit as shown in FIG. 14, the switching element 83 is in the switch position A in the initial phase 67b, so that the capacitor 62b is charged, in this case with a voltage profile which runs exponentially to a limit value. The initial phase 67b is ended after a predefined time, or on reaching a threshold value.

The switching element 83 is moved to the switch position C at the time 69b. In the first part 84 of the first phase 70b of transit oscillations of the resonant circuit, the current is oriented in the direction 76 and therefore runs via the path 82. In the second part 85 of the first phase 70b, the current 71b is oriented in the opposite direction, in the direction of the arrow 77, in which case the current can pass over the path 79, because the diode 80 is not reverse-biased. In addition, a portion of the current can pass over the path 82.

In the region of the second part 85 of the first phase 70b, the switching element 83 can be moved to a switch position B, in which case the current runs exclusively via the path 79 in the second part 85.

At the end of the first phase 70b at the time 72b, the voltage 68b reaches a maximum. Because the switching element 83 is in the switch position B and the diode 80 is reverse-biased, the resonant circuit 60b is, however, blocked. This blocked position is maintained for a third phase 86, in which the voltage 68b and the current 71b in any case change insignificantly. The switching element 83 is moved to the switch position A at the end of the third phase 86, at the time 87. In the subsequent second phase 73b, the capacitor is charged with a voltage profile which tends exponentially to a limit value. At the end of the third phase 73b at the time 88, the cycle period 75b formed the first phase 70b, the third phase 86 and the second phase 73b is complete, and a further cycle starts.

The use of the paths 82, 79 and the diode 80 allows the switching element 83 to be switched at any desired time within the part 85 of the first phase 70b, so that the switching need not take place exactly at a zero crossing of the current 71b, therefore contributing to simplification of the control circuit since there is no need to detect the zero crossing accurately. Furthermore, the diode 80 means that the oscillation is ended automatically at the time 72b, once all of the remaining energy in the resonant circuit 60b has been stored in the capacitor 62b again. The energy will have been dissipated during the first phase 70b, as is expressed in a voltage difference 89 between the maxima of the voltage 68b at the start of the first phase 70b and at the end of the first phase 70b.

The illustrated circuit makes it possible to produce high flux densities, high rates of change and a multiplicity of pulse flanks. Energy is saved and the amount of heat developed is reduced because it is not necessary to supply all the pulse energy to produce the next pulse but only the dissipated energy ΔW=ΔU_{c} 0.5 C U² which was lost during the first phase 70b. This makes it possible to produce considerably more pulses before the temperature of the coil or of the working area has risen to 41° C. The entire thermal capacity of the coil must be made use of solely by those components of the current which are therapeutically most effective. If these advantages are all considered together, then the invention allows an effectiveness increase to be achieved of up to one hundred times or more, which in the end has the advantage of a drastically reduced application time, for the user.

A further advantage of the resonant circuit according to the invention is that the remaining energy can be stored virtually without any losses in the capacitor over a relatively long time period. The pulse repetition frequency can therefore be varied conveniently within wide limits and can be matched to the external conditions and therapeutic requirements just by lengthening or shortening the time duration of the third phase 86 without the remaining energy being significantly influenced by this.

The maximum possible pulse repetition frequency corresponds to the resonant frequency of the resonant circuit and is achieved when the third phase 86 is reduced to a time duration of 0, and the energy dissipated during the first phase 70b is supplied during the first phase 70b, so that there is no second phase 73b, either. For this purpose, the capacitor 62b is preferably supplied with a voltage via the voltage supply until the voltage 68b across the capacitor is positive. The voltage supply 65b can be adapted and controlled in an appropriate manner for this purpose. Since in some circumstances, operations such as this leads to increased heating of the therapeutic appliance, such operation is in some circumstances restricted to a predetermined time period although this may be acceptable if, for example, the therapeutic effect is intended to be produced only in the region of a defined body point.

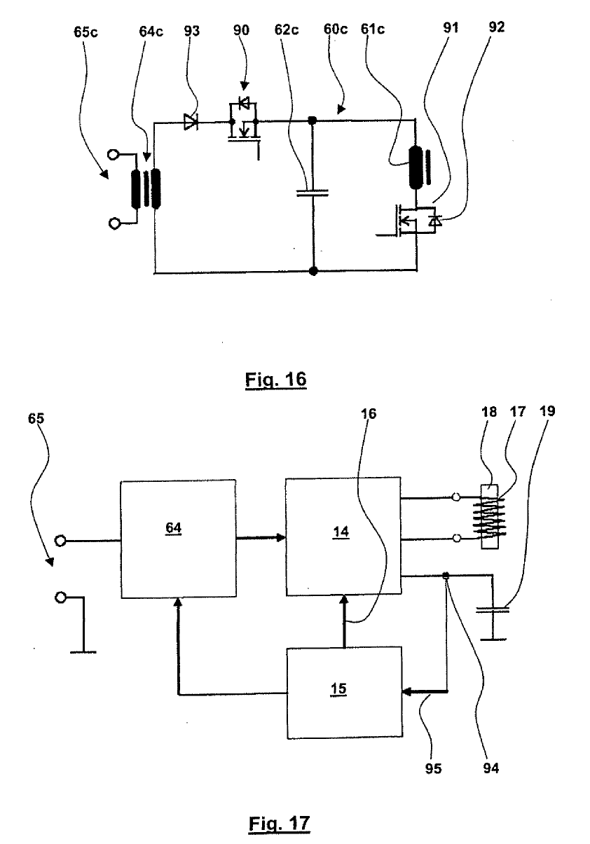

FIG. 16 shows a circuit in which the switch positions A, B, C of the switching element 83 are provided by means of MOSFET transistors 90, 91, as shown in FIG. 14. In this case, the resonant circuit 60c is formed by the capacitor 62c and the coil 61c as well as the MOSFET transistor 91, which in this case has the reverse-biased diode 92 instead of the diode 80. The high-voltage transformer 64c, the MOSFET transistor 90 and a diode 93 connected upstream of the MOSFET transistor 90 are connected in parallel with the capacitor 62c. The switch position A as shown in FIG. 14 accordingly results when the MOSFET transistor 90 is switched on, and the MOSFET transistor 91 is switched off. The switch position B results when the MOSFET transistor 90 is switched off and the MOSFET transistor 91 is switched off. The switch position C results when the MOSFET transistor 90 is switched off and the MOSFET transistor 91 is switched on. The diode 93 is required in order to prevent a discharge reverse flow from the capacitor 62c into the energy source.

It is optionally also possible to use IGBT transistors instead of the MOSFET transistors 90, 91. In this case, there is no need for the diode 93. If no IGBT with an integrated reverse-biased diode is used for the MOSFET transistor 91, this is additionally required as an individual component.

In order to achieve both a maximum flux density of 0.8 Tesla and major changes in the flux in practice, a capacitance of 4 microfarads of the capacitor 62 is charged to about 1,650 V. A capacitance such as this can be produced by:
using a single high-voltage capacitor with a high capacitance,
connecting a large number of low-capacitance high-voltage capacitors in parallel, or
using a bank of capacitors with a high capacitance and medium withstand voltage, connected in series and parallel.

In the case of a bank of capacitors with a high capacitance and a medium withstand voltage, self-healing metalized polypropylene capacitors can preferably be used instead of (bipolar) electrolytic capacitors, with the first-mentioned capacitors having a considerably lower internal resistance (ESR), thus resulting in low losses when using high pulse currents. A further advantage of the present types is that, in contrast to electrolytic capacitors, they do not suffer any loss of capacitance resulting from drying out, even after 10,000 operating hours. A loss of capacitance such as this could admittedly lead to an increase in the rate of change of the flux in a resonant circuit. However, the maximum flux density would be increasingly reduced at the same time.

The therapeutic appliance has control electronics which control the operation of the high-voltage transformer 64c and at the same time monitor the voltage across the capacitor 62c. This monitoring is carried out, for example, by means of a threshold-value circuit with hysteresis in order to recognize when a desired maximum capacitor voltage has been reached. When this threshold value has been reached, the energy transmission through the high-voltage transformer 64 is interrupted. Furthermore, a Schmitt triggered circuit is provided to identify the zero crossing of the capacitor voltage, in order to detect the time for switching from the switch position C to the B. A clock is also required for the entire procedure, in order to measure the waiting times between the individual pulses. A microcontroller is therefore preferably used for overall control.

The therapeutic appliance and the circuit in it preferably have technical data as follows, with the numerical values indicated in square brackets indicating preferred parameter values with a tolerance of ±15%.

| | | | |
|---|---|---|---|
| Maximum flux density | 0.1-2.0 | Tesla | [0.75] |
| “Bₘₐₓ”: | | | |
| Flank gradient “ΔB/Δt” | 0.3-8.0 | Tesla/millisecond | [0.88]B |
| at the zero crossing | | | |
| of the coil current | | | |
| Resonant frequency: | 150-5000 | Hz | [208] |
| Pulse repetition | 1-250 | hz | [10] |
| frequency (individual | | | |
| sinusoidal | | | |
| oscillations): | | | |
| Operating time for 10 | 4-10 | min | [4] |
| pulses per second | | | |
| before reaching 43° C. | | | |

Coil temperature (at an ambient temperature of 25° C.,
coil temperature measured on the surface)

| Capacitor capacitance: | 0.01-20 | microfarad | [4] |
|---|---|---|---|
| Coil inductance | 20-800 | millihenry | [146] |
| Maximum capacitor | 500-10 000 | volt | [1650] |
| voltage: | | | |
| Number of turns on the | 800-5000 | | [1700] |
| coil | | | |
| Mean power of the | 5-250 | watt | [8] |
| energy source | | | |
| Diameter of the iron | 5-30 | mm | [15] |
| powder core | | | |

1200 pulses ±15% can preferably be produced within two minutes by the described circuit, with each pulse containing one full cycle with
an increase from 0 to a maximum,
a drop from the maximum to 0,
a fall from 0 to a minimum, and
a further rise from the minimum to 0 of the current in the coil.

FIG. 17 shows a block diagram of an electrical circuit which corresponds essentially to the block diagram illustrated in FIG. 2. However, the power output stage 14 is preceded by the high-voltage transformer 64, which is in turn fed from the voltage source 65. As the measurement signal, the control electronics 15 receive a signal of the resonant circuit, in this case a signal from the capacitor, which is measured via a measurement element 94 or is tapped off, and is supplied via a line 95 to the control electronics 15. The control electronics act on the one hand on the power output stage 14 and on the other hand on the high-voltage transformer 64. For one particular refinement according to the invention, the current level for production of the peak value of the flux density of approximately 0.8 to 1 Tesla is reduced to below 20 A. In this case, by way of example, the coil has 1700 turns, and the magnet core of the coil is composed of an iron powder.

LIST OF REFERENCE SYMBOLS

| | |
|---|---|
| 1 | Therapeutic appliance |
| 2 | Plug |
| 3 | Module housing |
| 4 | Mains switch |
| 5 | Connection cable |
| 6 | Hand part |
| 7 | Working area |
| 8 | Control element |
| 9 | Display |
| 10 | Voltage supply |
| 11 | Pole |
| 12 | Pole |
| 13 | Electrical connection |
| 14 | Power output stage |
| 15 | Control electronics |
| 16 | Signal connection |
| 17 | Coil |
| 18 | Core |
| 19 | Capacitor |
| 20 | Circuit |
| 21 | Switching element |
| 22 | Load resistor |
| 23 | Diode |
| 24 | Induced voltage |
| 25 | Induced current |
| 26 | Supply voltage |
| 27 | Switching element |
| 28 | Switching element |
| 29 | Diode |
| 30 | Voltage |
| 31 | Voltage |
| 32 | Mains power supply voltage |
| 33 | Current |
| 34 | Voltage |
| 35 | Time |
| 36 | Phase |
| 37 | Phase |
| 38 | Phase |
| 39 | Phase |
| 40 | Phase |
| 41 | Switching element |
| 42 | Diode |
| 43 | Diode |
| 44 | Diode |
| 45 | Charging current |
| 46 | Oscillating current |
| 47 | Oscillating current |
| 48 | Oscillating current |
| 49 | Dissipated current |
| 50 | Control device |
| 51 | Method step |
| 52 | Method step |
| 53 | Method step |
| 54 | Method step |
| 55 | Method step |
| 56 | Method step |
| 57 | Method step |
| 58 | Method step |
| 59 | Resonant circuit |
| 60 | Resonant circuit |
| 61 | Coil |
| 62 | Capacitor |
| 63 | Switching element |
| 64 | High-voltage transformer |
| 65 | Voltage source |
| 66 | Switching element |
| 67 | Initial phase |
| 68 | Voltage capacitor |
| 69 | Time |
| 70 | First phase |
| 71 | Current coil |
| 72 | Time |
| 73 | Second phase |
| 74 | Time |
| 75 | Cycle period |
| 76 | Oscillating current |
| 77 | Oscillating current |
| 78 | Outgoer |
| 79 | Path |
| 80 | Diode |
| 81 | Outgoer |
| 82 | Path |
| 83 | Switching element |
| 84 | First part |
| 85 | Second part |
| 86 | Third phase |
| 87 | Time |
| 88 | Time |
| 89 | Voltage difference |
| 90 | MOSFET transistor |
| 91 | MOSFET transistor |
| 92 | Reverse diode |
| 93 | Diode |
| 94 | Outgoer |
| 95 | Line |

1.-41. (canceled)

42. A therapeutic appliance, having an electrical power supply, an electrical resonant circuit with a coil, and a working area,
with the electrical power supply being connected to at least one component in the resonant circuit,
wherein energy can be transferred via the connections to the at least one component in the resonant circuit, and a variable magnetic field which is produced in the coil passes through the working area,
a) wherein a control device is provided, by which at least one switching element can be operated via signal connections,
with the resonant circuit being closed in a first phase on operation of the at least one switching element,
with the resonant circuit being opened in a second phase on operation of the at least one switching element and an energy supply being enabled between the electrical power supply and the at least one component in the resonant circuit, and
b) wherein a means for determining a time to end the first phase being provided in the control device, from which the transient oscillations in the resonant circuit have not decayed below a predetermined extent.

43. The therapeutic appliance as claimed in claim 42, characterized in that at least one switching element can be operated by the control device via signal connections, with the resonant circuit being interrupted in a third phase on operation of the at least one switching element, and with the components in the resonant circuit being decoupled from the voltage supply.

44. The therapeutic appliance as claimed in claim 42, characterized in that
a) the resonant circuit has different paths for different current directions,
b) a component which blocks in one direction is arranged in a first path,
c) a switching element is arranged in the second path, and
d) the switching element can be operated via the control device at a time at which the transient oscillations pass the first path.

45. The therapeutic appliance as claimed in claim 42, characterized in that
a) the resonant circuit has one path for different current directions,
b) two switching elements are located in the path, and
c) each switching element can be operated for a respective current direction.

46. The therapeutic appliance as claimed in claim 42, characterized in that the power supply is provided via a high-voltage transformer.

47. The therapeutic appliance as claimed in claim 42, characterized in that a magnet core composed of a ferromagnetic iron powder passes through the coil.

48. The therapeutic appliance as claimed in claim 47, characterized in that the saturation flux density of the magnetic core is greater than 0.5 Tesla.

49. The therapeutic appliance as claimed in claim 47, characterized in that a control device and switching element are provided, with the control device being connected to the switching elements such that
a) an energy supply from the power supply to at least one component in the resonant circuit can be activated,
b) the energy supply from the power supply to the at least one component in the resonant circuit can be deactivated,
c) transient oscillations can be produced in the resonant circuit, and
d) the transient oscillations in the resonant circuit can be ended, and the energy can be dissipated from the resonant circuit via components which are arranged outside the resonant circuit.

50. The therapeutic appliance as claimed in claim 47, characterized in that a periodic electrical variable with a period duration T can be produced in the resonant circuit via the power supply and the control of switching elements by the control device.

51. The therapeutic appliance as claimed in claim 47, characterized in that a mechanical temperature switch is provided and interrupts the current supply to the coil at least temporarily when a limit temperature is exceeded.

52. The therapeutic appliance as claimed in claim 47, characterized in that a temperature sensor and a monitoring unit are provided, and the monitoring unit monitors whether the temperature sensed by the temperature sensor has exceeded a threshold value.

53. The therapeutic appliance as claimed in claim 47, characterized in that a load resistor is provided, is arranged physically separately from the working area, and via which energy can be dissipated from the resonant circuit.

54. The therapeutic appliance as claimed in claim 47, characterized in that energy can be fed back from the resonant circuit into a mains power supply system via a switching element.

55. The therapeutic appliance as claimed in claim 47, characterized in that a plurality of foil capacitors, which are connected to one another in series or in parallel, are used to form the capacitor in the resonant circuit.

56. The therapeutic appliance as claimed in claim 47, characterized in that MOSFET or IGBT transistors are used as the switching elements.

57. The therapeutic appliance as claimed in claim 47, characterized in that time control is provided in order to operate the switching elements in order to end transient oscillations in the resonant circuit.

58. The therapeutic appliance as claimed in claim 47, characterized in that
a) a monitoring device is provided in order to monitor the electrical signals of the power supply and/or of resonant circuit, and
b) based on the monitoring device, switching elements are operated in order to supply energy to the resonant circuit, in order to dissipate energy from the resonant circuit, and/or in order to end transient oscillations.
